(19) United States
(12) Patent Application Publication
(10) Pub. No.: US 2002/0192723 A1
(43) Pub. Date: Dec. 19, 2002

(54) Antigen to systemic lupus erythematosis and diagnostic assay

(76) Inventor: Tai June Yoo, Memphis, TN (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/513,145

(22) Filed: Feb. 25, 2000

Related U.S. Application Data
(60) Provisional application No. 60/121,548, filed on Feb. 25, 1999.

(51) Int. Cl. A61K 31/00; C12N 9/16; A01N 61/00; G01N 33/567; C12N 9/10; C07K 5/00; C07K 7/00; C07K 16/00; A61K 38/04; C07K 14/00; A61K 38/00; C12N 1/00; G01N 33/531; C07K 17/00

(52) U.S. Cl. 435/7.21; 530/326; 530/350; 530/403; 435/810; 435/193; 435/196; 436/543

(57) ABSTRACT

A 28 kD antigen present in the intracellular signal transduction oncogene and an immunoassay method for diagnosing Systemic Lupus Erythematosis using the 28 kD antigen as a target substance for detecting target binding substance in biological fluid from an animal or human having symptoms of Systemic Lupus Erythematosis.

### CROSS-REFERENCE TO RELATED APPLICATION

This application claims the benefit of co-pending U.S. provisional patent application Ser. No. 60/121,548, filed Feb. 25, 1999, which is incorporated by reference in its entirety herein.

### FIELD OF THE INVENTION

The present invention relates to the field of immunology and more specifically relates to the intracellular signal transduction oncogene and to an immunoassay method for detecting an autoimmune disease of the Systemic Lupus Erythematosis

### BACKGROUND OF THE INVENTION

In a prior study on Systemic Lupus Erythematosis and Autoantigen, Yoo et al. found that these patients have auto-antibody to c-raf protein. It was dominantly expressed in the stria vascularis, spiral ligament, spiral limbus and organ of Corti. A subsequent study showed that experimental animals injected with c-raf protein developed lymphadenopathy, skin lesions, abnormal gait, splenomegaly and hyperimmunoglobulinemia almost resembling a full blown autoimmune disease like Lupus Erythematosus. This information stirred interest whether the autoantibodies detected in the study are present in patients with Systemic Lupus Erythematosus.

Raf proteins are serine/threonine specific protein kinases which play a critical role in intracellular signaling downstream from many tyrosine kinase and G-protein-linked receptors. c-Raf binds to Ras in a GTP-dependent manner and signal transduction pathway involving this is frequently activated in tumor cells. This information added more interest in pursuing the study with SLE patients and perhaps other patients population.

### SUMMARY OF THE INVENTION

The present invention provides an isolated 28 kD antigen present in the intracellular signal transduction oncogene and an immunoassay method for diagnosing Systemic Lupus Erythematosis using the 28 kD antigen as a target substance for detecting target binding substance in biological fluid from an animal or human having symptoms of a disease of the inner ear.

Accordingly, it is an object of the present invention to provide an antigen identified in the intracellular signal transduction oncogene that reacts specifically with antibodies from the sera of patients having an autoimmune disease of the intracellular signal transduction oncogene.

It is a further object of the present invention to provide a simple, rapid, sensitive and reproducible method for detecting antibodies to antigens from the intracellular signal transduction oncogene.

It is a further object of the present invention to provide an isolated antigen from the intracellular signal transduction oncogene that reacts specifically with antibodies in the sera of patients having Systemic Lupus Erythematosis.

It is a further object of the present invention to provide a sensitive blood test for the detection of Systemic Lupus Erythematosis in the early stages of the disease.

It is a further object of the present invention to provide an immunoassay that can distinguish Systemic Lupus Erythematosis from other autoimmune ear diseases.

It is a further object of the present invention to provide an immunoassay that can monitor the progression of Systemic Lupus Erythematosis or the effects of treatment for Systemic Lupus Erythematosis.

It is a further object of the present invention to provide an antigen to be used for the immunotherapeutic treatment of Systemic Lupus Erythematosis.

These and other objects of the present invention will become apparent after reading the following detailed description of the disclosed embodiments and the appended claims.

### BRIEF DESCRIPTION OF THE DRAWING

The invention is illustrated in the drawings in which like reference characters designate the same or similar parts throughout the figures of which:

FIG. 1 is a Western blot demonstrating that GST-Raf-1 (SEQ.ID.NO.:4) is present in the membranous portion of the inner ear, but is not present in the neural portion of the inner ear, in facial nerve or in brain tissue.

### DETAILED DESCRIPTION OF THE INVENTION

Definitions

As used herein, the term “28 kD antigen” refers to a protein extract or peptide from the intracellular signal transduction oncogene of a mammal, a protein or peptide having the amino acid sequence N′-IVQQFGFQRRASDDGKLTQ-C′ (SEQ.ID.NO.:1) and antigenic variants thereof, a protein or peptide having the amino acid sequence N′-IVQQFGYQRRASDDGKLTD-C′ (SEQ.ID.NO.:2) and antigenic variants thereof and a recombinant GST-Raf-1 protein or peptide having the amino acid sequence N′-MEHIQGAWKTISNGFGFKDAVFDGSSCISPTIVQQFGYQRRASDDGKLTDPSK TSNTIRVFLPNKQRTVVNVRNGMSLHDCLMKALKVRGLQPECCAVFRLLHE HKGKKARLDWNTDAASLIGEELQVDFLDHVPLTTHNFARKTFLK-C′ (SEQ.ID.NO.:4) and antigenic variants thereof. All sequences referred to herein are shown in detail in the Sequence Listing attached hereto and incorporated herein.

As used herein, the term “target substance” refers to the 28 kD antigen or a nucleic acid molecule having a sequence encoding the 28 kD antigen of the intracellular signal transduction oncogene of a mammal.

As used herein, the term “immune sample” refers to samples having antibodies that interact specifically with the 28 kD antigen.

As used herein, the term “target-binding substance” refers to immune samples and to biological molecules, such as antibodies, which interact specifically with the 28 kD antigen. The term “target-binding substance” further include nucleic acid probes that hybridize under stringent hybridization conditions to a nucleic acid molecule having a sequence encoding the 28 kD antigen of the intracellular signal transduction oncogene of a mammal.

As used herein, the term “preimmune sample” refers to samples not having antibodies that interact specifically with the 28 kD antigen.

As used herein, the term “membranous structures” refers to the basilar membrane, organ of Corti, stria vascularis, spiral ligament and vestibular epithelium of the inner ear.

As used herein, the term “neural structures” refers to the spiral ganglion, cochlear nerve in the modiolus and vestibular nerve in the temporal bone of the inner ear.

As used herein, the term “GST-Raf-1” refers to recombinant purified GST-Raf-1 protein (SEQ.ID.NO.:4) of human Raf-1 protein (SEQ.ID.NO.:6). Anti-Raf-1 is obtained from Transduction Laboratories (Lexington, Ky.).

As used herein, the term “antigenic variant” refers to a protein or peptide having an amino acid sequence different from the protein or peptide to which it is compared, but having similar immunologic characteristics such as the ability to bind to one or more antibodies that bind to the protein or peptide to which it is compared.

As used herein, the term “antibody” includes, where appropriate, polyclonal antibodies, monoclonal antibodies, antibody fragments and mixtures of the foregoing.

### DESCRIPTION OF THE PREFERRED EMBODIMENT

An antigen and a diagnostic assay method for detecting antibodies to the antigen in a biological sample are described herein. The antigen is the 28 kD antigen, and the diagnostic assay method is specific for detecting antibodies to the 28 kD antigen. The 28 kD antigen is present in the membranous fraction of the inner ear, but is not present in the neural fraction of the inner ear, facial nerve or brain tissue (Suzuki et al., ORL, 59:10-17, 1997). The 28 kD antigen shows greater than 75% homology with residues 41 to 60 of Raf-1 protein from various species including, but not limited to, the human sequence (SEQ.ID.NO.:2) and the chicken sequence (SEQ.ID.NO.:3).

Preferably, the 28 kD antigen has the sequence of SEQ.ID.NO.:1, SEQ.ID.NO.:2, SEQ.ID.NO.:4 or antigenic variants thereof and reacts with a biological fluid from an individual having an autoimmune disease of the intracellular signal transduction oncogene. More preferably, the 28 kD antigen has the amino acid sequence of SEQ.ID.NO.:1 or SEQ.ID.NO.:2 or antigenic variants thereof and reacts with a biological fluid from an individual having Meniere's disease. Most preferably, the 28 kD antigen has the amino acid sequence of SEQ.ID.NO.:1 and antigenic variants thereof and reacts with a biological fluid from an individual having SLE.

The diagnostic assay for detecting the 28 kD antigen of the intracellular signal transduction oncogene can be, for example, an immunoassay. Such an immunoassay includes, but is not limited to, an ELISA, a Western blot assay, a competitive binding assay, a particle based immunoassay, a dual particle competitive immunoassay, a radioimmunoassay and any of the other immunoassay methods known to those skilled in the art.

For example, in a conventional immunoassay, such as an ELISA, an inert solid-phase material, usually a plastic microtiter plate, is contacted with a solution containing the target substance (28 kD antigen) so that the target substance binds to, or coats, the solid phase material. The bound target substance is then contacted with an aqueous sample obtained from an individual having symptoms of SLE, which may or which may not contain a target-binding substance (anti-28 kD antibody). Unbound target-binding substance is removed, and the amount of reacted target-binding substance is quantitated using any of a number of detection devices known to those skilled in the art. For example, the bound target-binding substance may be detected with a second antibody to which has been attached a detectable label such as an enzyme, radioisotope or fluorescent molecule.

The target substance for use in the present invention includes, but is not limited to, protein extracted from the intracellular signal transduction oncogene, fractions of protein extracted from the intracellular signal transduction oncogene, and an isolated 28 kD antigen extracted and purified from the intracellular signal transduction oncogene. In addition, the target substance for use in the present invention also includes, but is not limited to, a protein or peptide having the amino acid sequence of SEQ.ID.NO.:1 or antigenic variants thereof, a protein or peptide having the amino acid sequence of SEQ.ID.NO.:2 or antigenic variants thereof, and a protein or peptide having the amino acid sequence of SEQ.ID.NO.:4 or antigenic variants thereof obtained using recombinant DNA technology or synthesized by methods known to those skilled in the art of peptide synthesis. It will be understood by those skilled in the art that the term “amino acid sequence of SEQ.ID.NO.:1, 2, or 4” includes antigenic variants thereof.

The concentration of target substance for use in the present invention can range between approximately 1 μg/ml and 100 μg/ml. The more preferable range is between approximately 3 μg/ml and 50 μg/ml. The most preferable range is between approximately 5 μg/ml and 30 μg/ml. The target substance is dissolved in an aqueous solution and can be applied to an inert solid-phase support material by dipping, soaking, coating, spotting, spraying, blotting or other convenient means. Preferred methods include coating, spotting, spraying and blotting. More preferred methods include coating and blotting. For example, in an ELISA, a preferred volume for coating is between about 10 μl/well and 200 μl/well. a more preferred volume for coating is between about 30 μl/well and 150 μl/well. A most preferred volume for coating is between about 50 μl/well and 100 μl/well. Determination of the amount of target substance to be used for each method of application is well within the knowledge of one skilled in the art. For example, a standard target substance, target-binding substance assay combination can be used to determine the amount of target substance to be applied to the inert solid-phase support material.

The solvent for use in the present invention can be any solvent that can solubilize the target-binding substance, and that is sufficiently miscible with water to be completely removed by subsequent thorough rinsing with an aqueous solution. Such solvents include, but are not limited to phosphate buffered saline (PBS), tris(hydroxymethyl)amino methane (TRIS), N-2-hydroxyethylpeperazine-N′-2-ethanesulfonic acid (HEPES), citric acid-phosphate buffer and carbonate buffer. Such aqueous buffers and their appropriate pHs are well known to those skilled in the art. Mixtures of solvents may also be used. Preferred solvents include 0.1 M carbonate buffer, pH 9.0, and citric acid-phosphate buffer, pH 5.0. These solvents may contain other chemicals including, but not limited to, SDS, Tween-20, bromphenol blue, glycerol, dithiothreitol and the like.

The solid phase, or inert solid-phase support material, for use in the present invention can be in the form of, but is not limited to, a membrane, a bead, a microtiter plate or any other solid-phase support form known to those skilled in the art. Preferred forms include a membrane strip, a membrane well microtiter plate and a plastic well microtiter plate. More preferred forms include a membrane strip and a plastic well microtiter plate. A most preferred form is a plastic well microtiter plate. In addition, the inert solid-phase support material can be placed into a holder, including but not limited to, a membrane sheet holder, a dot-blot apparatus, a microtiter plate, a column, and a filter. Preferred holders include a membrane sheet holder, a dot-blot apparatus and a microtiter plate.

The blocking buffers for use in the present invention to prevent non-specific binding can be any suitable blocking buffer including, but not limited to, goat serum, fetal calf serum, gelatin, low fat milk, and Tween-20 at various dilutions in an aqueous solution.

The washing solution for use in the present invention can be any suitable aqueous buffer including, but not limited to, phosphate buffered saline (PBS), tris(hydroxymethyl)amino methane (TRIS) and N-2-hydroxy-ethylpeperazine-N′-2-ethanesulfonic acid (HEPES). Such aqueous buffers and their appropriate pHs are well known to those skilled in the art.

The target-binding substance for use in the present invention is a substance which binds specifically to the target substance. Examples of target-binding substances include, but are not limited to, antibodies (including monoclonal antibodies, polyclonal antibodies and mixtures thereof), antibody fragments and the like. Preferred target-binding substances are antibodies to proteins of the intracellular signal transduction oncogene. More preferred target-binding substances are antibodies to a 28 kD antigen of the membranous structure of the inner ear in serum from individuals having inner ear disease. Most preferred target-binding substances are antibodies to a 28 kD protein of the intracellular signal transduction oncogene in the serum of individuals having Systemic Lupus Erythematosis.

Any convenient indicator method can be used to detect binding of a target-binding substance to a target substance. Such methods include, but are not limited to, the use of enzymes, enzyme cofactors, enzyme effectors, chromogenic substances, fluorogenic substances, chemiluminescent substances, bioluminescent substances, labeled and radiolabeled antibodies. Preferred indicator methods are the peroxidase-labeled antibody method and the alkaline phosphatase-labeled antibody method.

The present invention further comprises an assay kit for detecting target-binding substance in a biological sample comprising an inert solid-phase support material having target-binding substance immobilized thereon and may further contain reagents and a holder for the inert solid-phase support material. The kit may additionally contain equipment for safely containing the samples, a vessel for containing the reagents, a timing means, and a calorimeter, reflectometer, or standard against which a color change may be measured. The reagents, including the target substance coated particle and the detectable particle are preferably lyophilized. Most preferably, the coated particle, and the detectable particle are provided in lyophilized form in a single container.

For example, an immunoassay kit useful for measuring the target-binding substance in a biological sample can involve a “sandwich immunoassay.” The kit contains a particle coated on its surface with the binding substance, a detectable particle capable of binding to the target-binding substance and a porous membrane having a pore size that prevents passage of the coated particle and allows passage of the detectable particle. The first step in the immunoassay is a binding step, and the second step is a detection step.

In the binding step, a solid phase particle or sphere coated with the target substance is combined in a solution with a sample containing the target-binding substance and reacted for a sufficient amount of time to allow the target substance and the target binding substance to interact. In the detection step, a detectable particle, such as a colored bead, coated with a substance that binds readily to the target-binding substance, such as protein A, protein G, a second antibody reactive to the target-binding substance, or a small synthetic affinity ligand is added to the suspension. The detectable particle binds to the target-binding substance complexed to the target substance coated particle. The reaction mixture is then placed on a membrane having a pore size of sufficient dimension to exclude passage of target substance coated particle which have bound target-binding substance and, therefore, bound detectable particle. Those components which are complexed as target substance particle plus target-binding substance plus detectable particle are retained on the membrane while the other components pass through the pores.

The complex is detected either visually with the naked eye or using a conventional detector, such as a calorimeter or reflectometer, well known to those skilled in the art. In this sandwich immunoassay, the presence of detectable particles indicates the presence of target-binding substance in the sample.

The present invention will be further described in connection with the following Examples, which are set forth for purposes of illustration only. Parts and percentages appearing in such Examples are by weight unless otherwise stipulated.

### EXAMPLES

Methods

Serum was obtained from patients with SLE. Sera from patients with a high double stranded DNA were collected from the Memphis Pathological Laboratory, a regional resource center in the Mid-Southern United States. A total of 33 sera were collected randomly with a high ds DNA (>2× the normal). Random computer selection guided the harvest of sera, and was done by the staff of Memphis Pathology Laboratory. The age group of the patients ranged from 18 to 81 years old, with a mean age of 31 and mostly women. The presence of the autoantibodies was investigated using the ELISA (enzyme-linked immunosorbent assay) technique.

Results

The 33 sera samples were studied for the presence of antibodies. Interestingly, 9 patients tested positive to c-Raf among 33 sampled. This would represent a 26% positive response rate. One out of 28 control patients showed positive.

Discussion

C-raf is an essential member of the growth factor ras pathway and a target for intervention strategies aimed at blocking cell proliferative responses. The presence of c-raf in SLE patients was detected at a rate of 26%.

### Example 1

### Isolation and Identification of 28 kD Protein from the Membranous Fraction of Retrocochlear Tissue

Protein Extraction

Proteins are extracted from the membranous fraction of retrocochlear tissue in accordance with the method of Suzuki, M. et al. ORL, 59:10-17, 1997 as follows. Briefly, forty Harley strain male guinea pigs (Sasco Co., Wilmington, Mass.) are anesthetized by intraperitoneal injection of a mixture of ketamine (70 mg/kg) and xylazine (70 mg/kg) and perfused with 0.01 M phosphate buffered saline (PBS), pH 7.4, through the left ventricle. Both temporal bones are removed and placed in crushed ice.

The membranous portion of the inner ear, including basilar membrane, organ of Corti, stria vascularis, spiral ligament and vestibular epithelium are dissected under a microscope. The neural portion of the inner ear, including the spiral ganglion, cochlear nerve in the modiolus, and vestibular nerve in the temporal bone, the facial nerve and brain tissue are dissected under a microscope.

The membranous tissue and the neural tissue are each sonicated for 20 seconds in lysis buffer (100 mM NaCl, 10 mM Tris buffer (Sigma Chemical Company, St. Louis, Mo.), pH 7.6; 1 mM ethylenediaminetetraacetate (EDTA; Sigma Chemical Company, St. Louis, Mo.), pH 8.0; a surfactant including, but not limited to. 0.1% sodium dodecyl sulfate (SDS, Sigma Chemical Company, St. Louis, Mo.) and 1% Nonidet P-40 (Sigma Chemical Company, St. Louis, Mo.; 2 μg/ml aprotinin; and, 100 mg/ml phenylmethylsulfonyl fluoride (PMSF, Sigma Chemical Company, St. Louis, Mo.; 2 μg/ml aprotinin; and, 100 mg/ml phenylmethylsulfonyl fluoride (PMSF, Sigma Chemical Company). The extracted membranous proteins and the extracted neural proteins are incubated at 0° C. for 30 minutes and centrifuged at 10,000 rpm for 10 minutes. Each supernatant is filtered through a 0.22 nun filter (Millipore Co., Bedford, Mass.), boiled for 5 minutes in a boiling water bath and stored at −80° C.

Protein concentration is determined after electrophoresis in one-dimensional 12% SDS-polyacrylamide gels (SDS-PAGE) using molecular weight standards (Life Technology, Inc., Grand Island, N.Y.). A range of 1 μg/ml to 10 μMg/ml of standard is loaded on the same gel and is compared with inner ear protein extract and with Raf-1 protein.

SDS-polyacrylamide Gel Electrophoresis (SDS-PAGE)

Samples are fractionated by SDS-PAGE using a 12% running gel and a 5% stacking gel in accordance with the known method of Laemmeli et al. Samples of membranous proteins, of neural proteins and of Raf-1 protein are each mixed 1:1 with 100 mM Tris, pH 6.8, 4% SDS, 0.2% bromphenol blue, 20% glycerol and 200 mM dithiothreitol and heated at 100° C. for three minutes. The gels are electrophoresed in a vertical electrophoresis apparatus (Life Technologies, Inc., Grand Island, N.Y.) at 90 volts for seven hours. The gels are fixed and stained with Coomassie brilliant blue (BioRad, Melville, N.Y.) and are destained with 45% methanol, 10% acetic acid and 45% distilled water. Apparent molecular weights of the separated components are calculated by comparison with prestained molecular weight markers (Life Technologies, Inc., Grand Island, N.Y.) electrophoresed in the same gel.

Western Blotting

Proteins, separated in 12% one-dimensional SDS-PAGE as described above, are electroblotted onto polyvinylidene difluoride (PVDF) membrane (BioRad, Melville, N.Y.) using a BioRad Semi-Dry Transblot Cell (BioRad, Melville, N.Y.) for 1 hour. The PVDF membrane is washed one time with 20 mM Tris, pH 7.5, 500 mM NaCl containing 0.025% Tween-20 (TTBS).

Amino Acid Sequencing

Samples separated in SDS-PAGE and are electroblotted onto PVDF membrane as described above. The band corresponding to the molecular weight of 28 kD is excised from the PVDF membrane and is microsequenced using automated Edman degradation (Applied Biosystems, Ragweed City, Calif.). Nineteen amino acids, having the sequence N′-IVQQFGFQRRASDDGKLTQ-C′ (SEQ.ID.NO.:1), are identified with an initial yield of approximately 78.6 picomoles.

Sequence comparison with the Swiss Protein Database shows the microsequence of the 28 kD membranous inner ear protein to have 89.5% identity, 19 amino acid overlap, with residues 41-60 of human Raf-1 protein (SEQ.ID.NO.:2) and 78.9% identity with residues 41-60 of chicken Raf-1 protein (SEQ.ID.NO.:3) as shown in Table 1.

TABLE 1
The 28 kD protein microsequence from guinea pig
inner ear and residues 41-60 of
Raf-1 protein from human and chicken

| Amino Acid | 40 | 50 | Identity |
|---|---|---|---|
| 28kD protein | IVQQFGFQR | RASDDGRLTQ | |
| Human Raf-1 | Y | D | 89.5% |
| Chick Raf-1 | Y | ISD | 78.9% |

### Example 2

### Preparation of Recombinant GST-Raf-1

A glutathione-s-transferase (GST)-Raf-1 construct, N-domain with residues of 1-149, (SEQ.ID.NO.:4) is provided by Dr. Mark Marshall (Indiana University, IN).

INFORMATION FOR SEQ ID NO:4:

(i) SEQUENCE CHARACTERISTICS:

(A) LENGTH: 148 amino acids

(B) TYPE: amino acid

(C) STRANDEDNESS: single

(D) TOPOLOGY: linear

(ii) MOLECULE TYPE: peptide

(iii) HYPOTHETICAL: NO

(iv) ANTI-SENSE: NO

(v) FRAGMENT TYPE: N-terminal

(vi) ORIGINAL SOURCE:

(A) ORGANISM: Homo sapiens

SEQUENCE DESCRIPTION: SEQ ID NO:4:

```
Met Glu His Ile Gln Gly Ala Trp Lys Thr Ile Ser Asn Gly Phe Gly
1 5 10 15
Phe Lys Asp Ala Val Phe Asp Gly Ser Ser Cys Ile Ser Pro Thr Ile
              20 25 30
Val Gln Gln Phe Gly Tyr Gln Arg Arg Ala Ser Asp Asp Gly Lys Leu
        35 40 45
Thr Asp Pro Ser Lys Thr Ser Asn Thr Ile Arg Val Phe Leu Pro Asn
    50 55 60
Lys Gln Arg Thr Val Val Asn Val Arg Asn Gly Met Ser Leu His Asp
65 70 75 80
Cys Leu Met Lys Ala Leu Lys Val Arg Gly Leu Gln Pro Glu Cys Cys
                85 90 95
Ala Val Phe Arg Leu Leu His Glu His Lys Gly Lys Lys Ala Arg Leu
              100 105 110
Asp Trp Asn Thr Asp Ala Ala Ser Leu Ile Gly Glu Glu Leu Gln Val
        115 120 125
Asp Phe Leu Asp His Val Pro Leu Thr Thr His Asn Phe Ala Arg Lys
    130 135 140
Thr Phe Leu Lys
145
```

An overnight culture is diluted 1:100 in fresh LB medium containing 100 μg/ml ampicillin and grown at 37° C. to an OD₆₀₀ of 0.5. The culture is equilibrated to 30° C. and 1 mM isopropyl-beta-D-thiogalacto-pyranoside (Sigma Chemical Co., St. Louis, Mo.) is added to induce GST-Raf-1 expression. After three hours of induction, the cells are pelleted, resuspended in PBS buffer, and incubated overnight at −70° C. The lysate is sonicated and the clear lysate is tumbled with glutathione-Sepharose beads (Pharmacia Biotech Inc., Piscataway, N.J.) for 30 minutes. The beads are washed four times with PBS and the GST-Raf-1 fusion protein is eluted with 10 MM reduced glutathione at pH 8.0. The GST-Raf-1 fusion protein is dialyzed against Tris-HCl buffer, pH 8.0, aliquoted, snap frozen, and stored at −80° C. The purity and concentration of the GST-Raf-1 fusion protein is determined by 12% SDS-PAGE.

### Example 3

### Western Blot Immunochemistry Of Sera From Patients With Inner Ear Disease

Proteins are extracted from the guinea pig inner ear, separated in SDS-PAGE and electroblotted onto PVDF membrane as in Example 1. The PVDF membranes are washed one time with TTBS.

Sera from patients with diseases, including Systemic Lupus Erythematosis, (experimental sera) are provided by the Department of Pathology, State University of Tennessee, Memphis, Tenn.) and Y. Yazawa (Department of Pathology, Siga University of Medical Science, Seta, Japan). The diagnosis of Meniere's disease is based on the AAO-HNS criteria. Sera from patients with no inner ear diseases (control sera) is obtained from age matched patients. Sera are stored at −20° C.

For use with serum as target binding substance (1st antibody), nonspecific binding is blocked using 25% goat serum in TTBS containing 0.02% sodium azide for one hour at room temperature.

### Example 4

### Presence of GST-Raf-1 (SEQ.ID.NO.:4) In Neural Inner Ear Proteins, Facial Nerve Proteins and Brain Tissue

Proteins extracted from the membranous portion of the inner ear, the neural portion of the inner ear, the facial nerve and the brain are electrophoresed and electroblotted onto PVDF membrane as in Example 1.

Anti-Raf-1 specific monoclonal antibody is obtained from Transduction Laboratories (Lexington, Ky.). This anti-Raf-1 recognizes amino acids 162-378 of human Raf-1 protein (SEQ.ID.NO.:5).

INFORMATION FOR SEQ ID NO:5:

(i) SEQUENCE CHARACTERISTICS:

(A) LENGTH: 217 amino acids

(B) TYPE: amino acid

(C) STRANDEDNESS: single

(D) TOPOLOGY: linear

(ii) MOLECULE TYPE: peptide

(iii) HYPOTHETICAL: NO

(iv) ANTI-SENSE: NO

(v) FRAGMENT TYPE: internal

(vi) ORIGINAL SOURCE:

(A) ORGANISM: Homo sapiens

(xi) SEQUENCE DESCRIPTION: SEQ ID NO:5:

```
Gly Phe Arg Cys Gln Thr Cys Gly Tyr Lys Phe His Glu His Cys Ser
1 5 10 15
Thr Lys Val Pro Thr Met Cys Val Asp Trp Ser Asn Ile Arg Gln Leu
              20 25 30
Leu Leu Phe Pro Asn Ser Thr Ile Gly Asp Ser Gly Val Pro Ala Leu
        35 40 45
Pro Ser Leu Thr Met Arg Arg Met Arg Glu Ser Val Ser Arg Met Pro
    50 55 60
Val Ser Ser Gln His Arg Tyr Ser Thr Pro His Ala Phe Thr Phe Asn
65 70 75 80
Thr Ser Ser Pro Ser Ser Glu Gly Ser Leu Ser Gln Arg Gln Arg Ser
                    85 90 95
Thr Ser Thr Pro Asn Val His Met Val Ser Thr Thr Leu Pro Val Asp
              100 105 110
Ser Arg Met Ile Glu Asp Ala Ile Arg Ser His Ser Glu Ser Ala Ser
          115 120 125
Pro Ser Ala Leu Ser Ser Ser Pro Asn Asn Leu Ser Pro Thr Gly Trp
    130 135 140
Ser Gln Pro Lys Thr Pro Val Pro Ala Gln Arg Glu Arg Ala Pro Val
145 150 155 160
Ser Gly Thr Gln Glu Lys Asn Lys Ile Arg Pro Arg Gly Gln Arg Asp
                  165 170 175
Ser Ser Tyr Tyr Trp Glu Ile Glu Ala Ser Glu Val Met Leu Ser Thr
              180 185 190
Arg Ile Gly Ser Gly Ser Phe Gly Thr Val Tyr Lys Gly Lys Trp His
          195 200 205
Gly Asp Val Ala Val Lys Ile Leu Lys
    210 215
```

SEQUENCE DESCRIPTION: SEQ ID NO:6:

(i) SEQUENCE CHARACTERISTICS:

(A) LENGTH: 648 amino acids

(B) TYPE: amino acid

(C) STRANDEDNESS: single

(D) TOPOLOGY: linear

(ii) MOLECULE TYPE: protein

(iii) HYPOTHETICAL: NO

(iv) ANTI-SENSE: NO

(v) FRAGMENT TYPE: N-terminal

(vi) ORIGINAL SOURCE:

(A) ORGANISM: Homo sapiens

(xi) SEQUENCE DESCRIPTION: SEQ ID NO:6:

```
Met Glu His Ile Gln Gly Ala Trp Lys Thr Ile Ser Asn Gly Phe Gly
1 5 10 15
Phe Lys Asp Ala Val Phe Asp Gly Ser Ser Cys Ile Ser Pro Thr Ile
            20 25 30
Val Gln Gln Phe Gly Tyr Gln Arg Arg Ala Ser Asp Asp Gly Lys Leu
        35 40 45
Thr Asp Pro Ser Lys Thr Ser Asn Thr Ile Arg Val Phe Leu Pro Asn
    50 55 60
Lys Gln Arg Thr Val Val Asn Val Arg Asn Gly Met Ser Leu His Asp
65 70 75 80
Cys Leu Met Lys Ala Leu Lys Val Arg Gly Leu Gln Pro Glu Cys Cys
                85 90 95
Ala Val Phe Arg Leu Leu His Glu His Lys Gly Lys Lys Ala Arg Leu
              100 105 110
Asp Trp Asn Thr Asp Ala Ala Ser Leu Ile Gly Glu Glu Len Gln Val
        115 120 125
Asp Phe Leu Asp His Val Pro Leu Thr Thr His Asn Phe Ala Arg Lys
    130 135 140
Thr Phe Leu Lys Leu Ala Phe Cys Asp Ile Cys Gln Lys Phe Leu Leu
145 150 155 160
Asn Gly Phe Arg Cys Gln Thr Cys Gly Tyr Lys Phe His Glu His Cys
                  165 170 175
Ser Thr Lys Val Pro Thr Met Cys Val Asp Trp Ser Asn Ile Arg Gln
              180 185 190
Leu Leu Leu Phe Pro Asn Ser Thr Ile Gly Asp Ser Gly Val Pro Ala
        195 200 205
Leu Pro Ser Leu Thr Met Arg Arg Met Arg Glu Ser Val Ser Arg Met
    210 215 220
Pro Val Ser Ser Gln His Arg Tyr Ser Thr Pro His Ala Phe Thr Phe
225 230 235 240
Asn Thr Ser Ser Pro Ser Ser Glu Gly Ser Leu Ser Gln Arg Gln Arg
                  245 250 255
Ser Thr Ser Thr Pro Asn Val His Met Val Ser Thr Thr Leu Pro Val
              260 265 270
Asp Ser Arg Met Ile Glu Asp Ala Ile Arg Ser His Ser Glu Ser Ala
        275 280 285
Ser Pro Ser Ala Leu Ser Ser Ser Pro Asn Asn Leu Ser Pro Thr Gly
    290 295 300
Trp Ser Gln Pro Lys Thr Pro Val Pro Ala Gln Arg Glu Arg Ala Pro
305 310 315 320
Val Ser Gly Thr Gln Glu Lys Asn Lys Ile Arg Pro Arg Gly Gln Arg
                  325 330 335
Asp Ser Ser Tyr Tyr Trp Glu Ile Glu Ala Ser Glu Val Met Leu Ser
              340 345 350
Thr Arg Ile Gly Ser Gly Ser Phe Gly Thr Val Tyr Lys Gly Lys Trp
        355 360 365
His Gly Asp Val Ala Val Lys Ile Leu Lys Val Val Asp Pro Thr Pro
    370 375 380
Glu Gln Phe Gln Ala Phe Arg Asn Glu Val Ala Val Leu Arg Lys Thr
385 390 395 400
Arg His Val Asn Ile Leu Leu Phe Met Gly Tyr Met Thr Lys Asp Asn
                  405 410 415
Leu Ala Ile Val Thr Gln Trp Cys Glu Gly Ser Ser Leu Tyr Lys His
              420 425 430
Leu His Val Gln Glu Thr Lys Phe Gln Met Phe Gln Leu Ile Asp Ile
        435 440 445
Ala Arg Gln Thr Ala Gln Gly Met Asp Tyr Leu His Ala Lys Asn Ile
    450 455 460
Ile His Arg Asp Met Lys Ser Asn Asn Ile Phe Leu His Glu Gly Leu
465 470 475 480
Thr Val Lys Ile Gly Asp Phe Gly Leu Ala Thr Val Lys Ser Arg Trp
                  485 490 495
Ser Gly Ser Gln Gln Val Glu Gln Pro Thr Gly Ser Val Leu Trp Met
              500 505 510
Ala Pro Glu Val Ile Arg Met Gln Asp Asn Asn Pro Phe Ser Phe Gln
        515 520 525
Ser Asp Val Tyr Ser Tyr Gly Ile Val Leu Tyr Glu Leu Met Thr Gly
    530 535 540
Glu Leu Pro Tyr Ser His Ile Asn Asn Arg Asp Gln Ile Ile Phe Met
545 550 555 560
Val Gly Arg Gly Tyr Ala Ser Pro Asp Leu Ser Lys Leu Tyr Lys Asn
                  565 570 575
Cys Pro Lys Ala Met Lys Arg Leu Val Ala Asp Cys Val Lys Lys Val
              580 585 590
Lys Glu Glu Arg Pro Leu Phe Pro Gln Ile Leu Ser Ser Ile Glu Leu
        595 600 605
Leu Gln His Ser Leu Pro Lys Ile Asn Arg Ser Ala Ser Glu Pro Ser
    610 615 620
Leu His Arg Ala Ala His Thr Glu Asp Ile Asn Ala Cys Thr Leu Thr
625 630 635 640
Thr Ser Pro Arg Leu Pro Val Phe
                  645
```

For use with anti-Raf-1 as target-binding substance (1st antibody), nonspecific binding is blocked using 5% nonfat dry milk in 10 mM Tris, pH 7.5, 100 mM NaCl and 0.1% Tween-20 for one hour at room temperature. The PVDF membrane is incubated in anti-Raf-1 diluted 1:1000 in 5% nonfat dry milk in 10 mM Tris, pH 7.5, 100 mM NaCl and 0.1% Tween-20 overnight at room temperature. The membrane is then washed three times in TTBS and incubated with peroxidase-conjugated goat anti-mouse IgG (Sigma Chemical Co., St. Louis, Mo.). Immunoreactive bands are visualized using 0.05 M Tris-HCl, pH 7.6, containing 0.02% 3,3′-diamino-benzidine (Chemicon International, Inc., Temecula, Calif.) and 0.01% hydrogen peroxide.

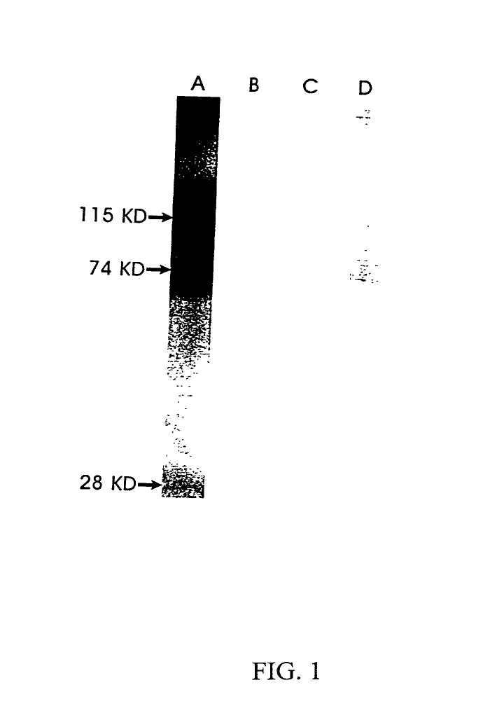

Anti-Raf-1 monoclonal antibody recognizes 115 kD and 74 kD proteins in extracts from the membranous portion of the inner ear (FIG. 1, Col. A), the neural portion of the inner ear FIG. 1, Col. B), the facial nerve (FIG. 1, Col. C) and the brain (FIG. 1, Col. D). In contrast, anti-Raf-1 monoclonal antibody recognizes the 28 kD protein (SEQ.ID.NO.:1) in extracts from the membranous portion of the inner ear, but not in extracts from the neural portion of the inner ear, the facial nerve and the brain.

### Example 5

### Reactivity of Sera From Systemic Lupus Erythematosis Patients with GST Raf-1 (SEQ.ID.NO.:4)

Sera from 33 Systemic Lupus Erythematosis patients, provided as in Example 5, are assayed for antibodies against GST-RAF-1 (SEQ.ID.NO.:4) in ELISA.

One hundred microliters of GST-Raf-1 (SEQ.ID.NO.:4) containing 5 μg/μl in 0.1 M carbonate buffer, pH 9.6, are dispensed into each well of a polystyrene microtiter plate (Costa, Cambridge, Mass.) and incubated overnight at 4° C. The antigen coated plates are washed three times in PBS-0.05% Tween buffer and incubated with patient's sera (1:40, 1:80, or 1:160 dilution) or with 0.1 M carbonate buffer, pH 9.6, as a control, overnight at 4° C. The plates are washed five times with PBS-0.05% Tween buffer and incubated overnight with c-chain specific anti human IgG antibodies (Sigma chemical Co., St. Louis, Mo.) at 4° C. The plates are washed five times in PBS-0.05% Tween buffer and citric acid-phosphate buffer, pH 5.0, containing 0.15 mg/ml of o-phenylenediamine (Sigma Chemical Co., St. Louis, Mo.) is added. The color is developed at room temperature and the reaction is stopped by 2.5 M sulfuric acid. The color is measured at 492 nm.

As shown in Table 1, 9 out of 33 sera obtained from Systemic Lupus Erythematosis patients (26%) recognize GST-Raf-1 (SEQ.ID.NO.:4) in ELISA at dilutions of 1:40, 1:80 and 1:160. Seven of these sera are tested in Western blot analysis and show positive reactivity with the 28 kD band in membranous inner ear extracts of the guinea pig.

While the foregoing specification teaches the principles of the present invention, with examples provided for the purpose of illustration, it will be understood that the practice of the invention encompasses all of the usual variations, adaptation, modification or deletions as come within the scope of the following claims and their equivalents. All patents, applications, documents and publications referred to herein are incorporated by reference in their entirety.

claimed is:

1. An antigen of the intracellular signal transduction protein, wherein the antigen is reactive with antibodies from patients having Systemic Lupus Erythematosis.

2 The antigen of claim 1, wherein the antigen is a protein or peptide selected from the group consisting of proteins, proteins purified from extracts of membranous inner ear proteins, recombinant proteins or peptides and synthesized proteins or peptides.

3. The antigen of claim 1, wherein the antigen is a protein or peptide having an amino acid sequence selected from the group consisting of SEQ.ID.NO.:1, SEQ.ID.NO.:2, SEQ.ID.NO.:4 or antigenic variants of the foregoing, and mixtures of the foregoing.

4. The antigen of claim 1, wherein the antigen is a protein or peptide having an amino acid sequence selected from the group consisting of SEQ.ID.NO.:1 and SEQ.ID.NO.:2 or antigenic variants of the foregoing, and mixtures of the foregoing.

5. The antigen of claim 1, wherein the antigen is a protein or peptide having an amino acid sequence consisting of SEQ.ID.NO.:1 or antigenic variants thereof.

6. A method of detecting Systemic Lupus Erythematosis in an animal or human, comprising the steps of:
(a) incubating a biological sample from the animal or human with a target substance under conditions sufficient to bind a target-binding substance in the biological sample to the target substance, wherein the target substance is a 28 kD antigen or a nucleic acid molecule encoding a 28 kD antigen of the intracellular signal transduction oncogene of a mammal and/or recombinant proteins; and,
(b) detecting the target-binding substance bound to the target substance.

7. The method of claim 6, wherein the target substance is a protein or peptide having an amino acid sequence selected from the group consisting of SEQ.ID.NO.:1, SEQ.ID.NO.:2, SEQ.ID.NO.:4 or antigenic variants of the foregoing, and mixtures of the foregoing.

8. The method of claim 6, wherein the target substance is a protein or peptide having an amino acid sequence selected from the group consisting of SEQ.ID.NO.:1, SEQ.ID.NO.:2, or antigenic variants of the foregoing, and mixtures of the foregoing.

9. The method of claim 6, wherein the target substance is a protein or peptide having an amino acid sequence of SEQ.ID.NO.:1 or antigenic variants thereof.

10. The method of claim 6, wherein the target-binding substance is an antibody.

11. The method of claim 6, wherein the antibody is selected from the group consisting of polyclonal antibodies, monoclonal antibody, antibody fragments and mixtures thereof.

12. The method of claim 6, wherein the target-binding substance is a nucleic acid probe.

13. The method of claim 6, wherein the amount of target-binding substance in the biological sample is quantitated.

14. The method of claim 6, wherein the biological sample is a biological fluid from an animal or human having symptoms of Systemic Lupus Erythematosis.

15. The method of claim 6, wherein the biological sample is a biological fluid from a patient having symptoms of Systemic Lupus Erythematosis.

16. The method of claim 6, wherein the biological fluid is serum.

17. An assay kit for detecting Systemic Lupus Erythematosis in a patient, comprising:
(a) a solid phase having a 28 kD target substance bound thereto which reacts with a target-binding substance in a biological fluid from an animal or human having symptoms of Systemic Lupus Erythematosis; and,
(b) means for detecting binding of the target binding substance to the target substance.

18. The assay kit of claim 17, wherein the target substance is a protein or peptide selected from the group consisting of extracts of intracellular signal transduction oncogenes, fractions of intracellular signal transduction oncogenes, proteins or peptides purified from extracts of intracellular signal transduction oncogenes, recombinant proteins or peptides and synthesized proteins or peptides.

19. The assay kit of claim 17, wherein the target substance is a protein or peptide having an amino acid sequence selected from the group consisting of SEQ.ID.NO.:1, SEQ.ID.NO.:2 and SEQ.ID.NO.:4.


SEQUENCE LISTING (6 sequences)

SEQ ID NO: 1 - protein, 19 aa, Guinea Pig
IVQQFGFQRRASDDGKLTQ

SEQ ID NO: 2 - protein, 19 aa, Homo sapiens
IVQQFGYQRRASDDGKLTD

SEQ ID NO: 3 - protein, 19 aa, Chicken
IVQQFGYQRRASDDGKISD

SEQ ID NO: 4 - protein, 148 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLK

SEQ ID NO: 5 - protein, 217 aa, Homo sapiens
GFRCQTCGYKFHEHCSTKVPTMCVDWSNIRQLLLFPNSTIGDSGVPALPSLTMRRMRESVSRMPVSSQHRYSTPHAFTFNTSSPSSEGSLSQRQRSTSTPNVHMVSTTLPVDSRMIEDAIRSHSESASPSALSSSPNNLSPTGWSQPKTPVPAQRERAPVSGTQEKNKIRPRGQRDSSYYWEIEASEVMLSTRIGSGSFGTVYKGKWHGDVAVKILK

SEQ ID NO: 6 - protein, 648 aa, Homo sapiens [sequence omitted]